(19) United States
(12) Patent Application Publication
(10) Pub. No.: US 2006/0052289 A1
(43) Pub. Date: Mar. 9, 2006

(54) Use of somatostatin analogs in sleep apnea

(76) Inventors: Christian Bruns, Freiburg (DE); Peter Marbach, Thewil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/540,358

(22) PCT Filed: Dec. 30, 2003
(86) PCT No.: PCT/EP03/14971

§ 371 Date: Jun. 23, 2005

(30) Foreign Application Priority Data
Jan. 3, 2003 (GB) 0300095.7

(51) Int. Cl. A61K 38/31

(52) U.S. Cl. 514/9

(57) ABSTRACT

Somatostatin analogues, e.g. comprising an amino acid sequence of formula -(D/L)Trp-Lys-X₁-X₂- wherein each of X₁, and X₂ is an amino acid residue as disclosed in the specification, have interesting properties in the treatment of sleep apnea.

The present invention relates to a new use for somatostatin analogues.

Somatostatin is a tetradecapeptide having the structure

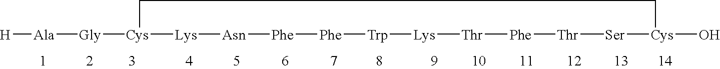

Somatostatin analogues of particular interest have been described e.g. in WO 97/01579. Said somatostatin analogues comprise the amino acid sequence of formula I
-(D/L)Trp-Lys-X₁-X₂- I
wherein X₁ is a radical of formula (a) or (b) 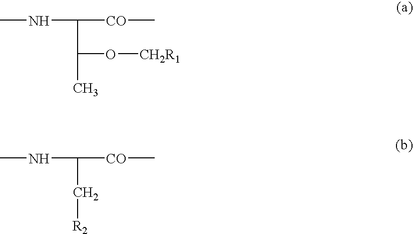
wherein R₁ is optionally substituted phenyl, wherein the substituent may be halogen, methyl, ethyl, methoxy or ethoxy, 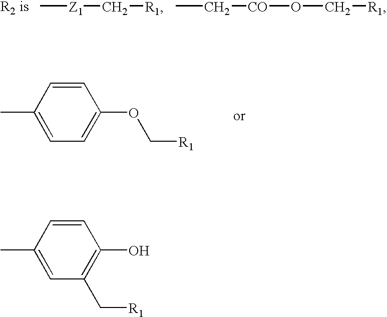
wherein Z₁ is O or S, and
X₂ is an α-amino acid having an aromatic residue on the C_{α} side chain, or an amino acid unit selected from Dab, Dpr, Dpm, His, (Bzl)HyPro, thienyl-Ala, cyclohexyl-Ala and t-butyl-Ala, the residue Lys of said sequence corresponding to the residue Lys⁹ of the native somatostatin-14, in free form, in salt form or in protected form.

By somatostatin analogue as used herein is meant a straight-chain or cyclic peptide derived from that of the naturally occurring somatostatin-14, comprising the sequence of formula I and wherein additionally one or more amino acid units have been omitted and/or replaced by one or more other amino acid radical(s) and/or wherein one or more functional groups have been replaced by one or more other functional groups and/or one or more groups have been replaced by one or several other isosteric groups. In general the term covers all modified derivatives of the native somatostatin-14 comprising the above sequence of formula I which have binding affinity in the nM range to at least one somatostatin receptor subtype as defined hereinafter.

Another example of somatostatin analogue for use according to the invention is 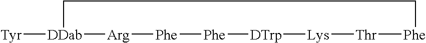
also known under the name KE108, in free form or in salt form, e.g. a pharmaceutically acceptable salt form, e.g. as indicated below.

These compounds are referred to hereinafter as compounds of the invention.

Preferably, the somatostatin analogue is an analogue in which the residues at positions 8 through 11 of the somatostatin-14 are represented by the sequence of formula I as defined above.

More preferably, the somatostatin analogue is an analogue as disclosed above comprising a hexapeptide unit, the residues at positions 3 through 6 of said hexapeptide unit comprising the sequence of formula I. Particularly preferred is a somatostatin hexapeptide wherein the residues at positions 1 and 2 of the hexapeptide unit may be any of those as known in the art, e.g. as disclosed by A. S. Dutta in Small Peptides, Vol. 19, 292-354, Elsevier, 1993, or as substituents for, Phe⁶ and/or Phe⁷ of somatostatin-14.

More particularly the somatostatin analogue is an analogue in which the hexapeptide unit is cyclic, e.g. having a direct peptide linkage between the α-carbonyl group of the residue at position 6 and the α-amino group of the residue at position 1.

While Lys, X₁ and X₂ in the sequence of formula I have the L-configuration, Trp may have the D- or L-configuration. Preferably Trp has the D-configuraton.

X₁ is preferably a residue of formula (a) or (b), R₂ being preferably

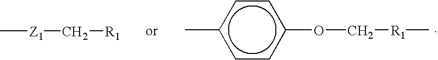

When X₂ comprises an aromatic residue on the C_{α} side chain, it may suitably be a natural or unnatural α-amino acid, e.g. Phe, Tyr, Trp, Nal, Pal, benzothienyl-Ala, Tic and thyronin, preferably Phe or Nal, more preferably Phe. X₂ is preferably an α-amino acid bearing an aromatic residue on the C_{α} side chain.

When R₁ is substituted phenyl, it may suitably be substituted by halogen, methyl, ethyl, methoxy or ethoxy e.g. in ortho and/or para. More preferably R₁ is unsubstituted phenyl.

Z₁ is preferably O.

Representative compounds of the invention are e.g. compounds of formula (II) 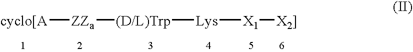
wherein
X₁ and X₂ are as defined above,
A is a divalent residue selected from Pro, 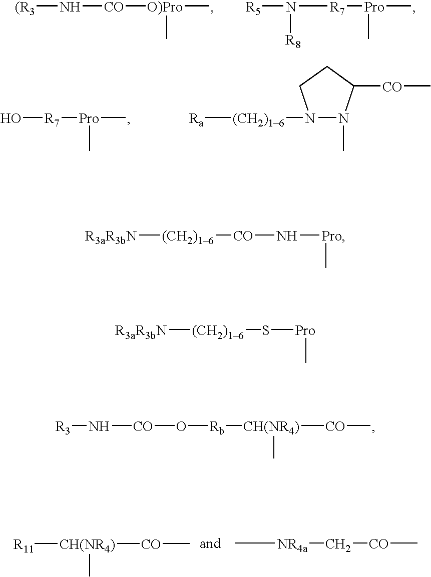
   wherein R₃ is NR₈R₉—C₂₋₆alkylene, guanidino-C₂₋₆alkylene or C₂₋₆alkylene-COOH, R₃ₐ is H, C₁₋₄alkyl or has independently one of the significances given for R₃, R_{3b} is H or C₁₋₄alkyl, Rₐ is OH or NR₅R₆, Rb is —(CH₂)₁, or —CH(CH₃)—, R₄ is H or CH₃, R₄ₐ is optionally ring-substituted benzyl, each of R₅ and R₆ independently is H, C₁₋₄alkyl, co-amino-C₁₋₄alkylene, ω-hydroxy-C₁₋₄alkylene or acyl, R₇ is a direct bond or C₁₋₆-alkylene, each of R₈ and R₉ independently is H, C, alkyl, ω-hydroxy-C₂₋₄alkylene, acyl or CH₂OH—(CHOH)_{c}—CH₂— wherein c is 0, 1, 2, 3 or 4, or R₈ and R₉ form together with the nitrogen atom to which they are attached a heterocyclic group which may comprise a further heteroatom, and R₁₁, is optionally ring-substituted benzyl, —(CH₂)₁₄—OH, CH₃—CH(OH)— or —(CH₂)₁₋₅—NR₅R₆, and
ZZₐ is a natural or unnatural α-amino acid unit.

ZZₐ may have the D- or L-configuration. When ZZₐ is a natural or unnatural α-amino acid unit, it may suitably be e.g. Thr, Ser, Ala, Val, Ile, Leu, Nle, His, Arg, Lys, Nal, Pal, Tyr, Trp, optionally ring-substituted Phe or N-benzyl-Gly. When ZZₐ is Phe, the benzene ring thereof may be substituted by e.g. NH₂, NO₂, CH₃, OCH₃ or halogen, preferably in para position. When ZZₐ is Phe, the benzene ring thereof is preferably unsubstituted.

When A comprises a Pro amino acid residue, any substituent present on the proline ring, e.g. R₃—NH—CO—O— etc., is preferably in position 4. Such substituted proline residue may exist in the cis form, e.g. 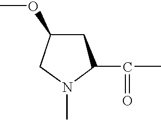
as well as in the trans form. Each geometric isomer individually as well as mixtures thereof are compounds of the invention.

When A is 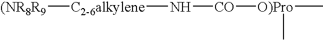
where NR₈R₉ forms a heterocyclic group, such group may be aromatic or saturated and may comprise one nitrogen or one nitrogen and a second heteroatom selected from nitrogen and oxygen. Preferably the heterocyclic group is e.g. pyridyl or morpholino. C₂-Alkylene in this residue is preferably —CH₂—CH₂—.

Any acyl as R₅, R₆, R and R₉ in A may be e.g. R₁₂CO— wherein R₁₂ is H, C₁₋₄alkyl, C₂₋₄alkenyl, C₃₋₄cycloalkyl or benzyl, preferably methyl or ethyl. When R₄ₐ or R₁₁ in A is ring-substituted benzyl, the benzene ring may be substituted as indicated above for ZZₐ.

Particularly preferred are compounds of formula III 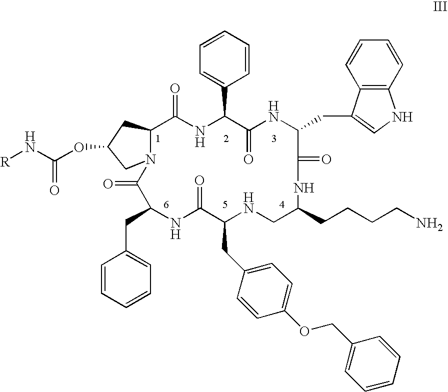
wherein R is NR₁₀R₁₁—C₂₋₆alkylene or guanidine-C₂₋₆alkylene, and each of R₁₀ and R₁₁ independently is H or C₁₋₄alkyl,
in free form, in salt form or protected form.

Preferably R is NR₁₀R₁₁—C₂₋₆alkylene. Preferred compounds of formula III are the compounds wherein R is 2-amino-ethyl, namely cyclo[{4-(NH₂—C₂H₄—NH—CO—O—)Pro}-Phg-DTrp-Lys-Tyr(4-Bzl)-Phe] (referred herein to as Compound A) in free form, salt form or protected form. Phg means —HN—CH(C₆H₅)—CO— and Bzl means benzyl.

A compound of the invention in protected form corresponds to a somatostatin analogue wherein at least one of the amino groups is protected and which by deprotection leads to a compound of formula II, preferably physiologically removable. Suitable amino protecting groups are e.g. as disclosed in “Protective Groups in Organic Synthesis”, T. W. Greene, J. Wiley & Sons NY (1981), 219-287, the contents of which being incorporated herein by reference. Example of such an amino protecting group is acetyl.

The compounds of the invention, e.g. Compound A, may exist e.g. in free or salt form. Salts include acid addition salts with e.g. inorganic acids, polymeric acids or organic acids, for example with hydrochloric acid, acetic acid, lactic acid, aspartic acid, benzoic acid, succinic acid or pamoic acid. Acid addition salts may exist as mono- or divalent salts, e.g. depending whether 1 or 2 acid equivalents are added to the compound of the invention in free base form. Preferred salts are the lactate, aspartate, benzoate, succinate and pamoate including mono- and di-salts, more preferably the aspartate di-salt and the pamoate monosalt, e.g. of Compound A.

Compound A and its salts are disclosed e.g. in WO02/10192, the contents of which being incorporated herein by reference.

The compounds of the invention have, on the basis of observed activity, e.g. inhibition of growth hormone, been found to be useful e.g. in the treatment of acromegaly.

It has now been found that the compounds of the invention, e.g. Compound A, have a beneficial relief effect on sleep apnea and promotes paradoxical sleep.

Sleep apnea is recognised as a significant cause of morbidity and mortality. It is defined as absence of airflow for greater than ten seconds and can be classified into three types: obstructive, central, and mixed. In central apnea, airflow and respiratory movements temporarily cease, owing to disordered central regulation of respiration. In obstructive apnea, thoracic and abdominal respiratory efforts continue, but there is no effective airflow. Some apneic periods begin with a central process and then become obstructive and therefore are mixed apneas. Many persons with sleep apnea have obstructive, central, and mixed events. Some patients also manifest hypopnea, which is decreased tidal volume with associated oxygen desaturation. Apnea termination is usually accompanied by evidence of arousal on the sleep EEG, which often is not appreciated consciously by the patient. The frequency and duration of apneas are variable between patients, but a typical patient may have as many as 300 apneas per night. The obstructive form is more common than the central form.

Symptoms are related to the length and frequency of apneic or hypopneic episodes, oxygen desaturation, and to whether the syndrome is predominantly obstructive or central. Obstructive sleep apnea is usually characterized by excessive sleepiness. Somnolence may occur at inopportune times, such as during conversations, while eating, during work, or driving. Excessive somnolence is the most constant symptom, but in some patients depression, intellectual deterioration, personality change, anxiety, memory disturbance, early morning confusion, deterioration of judgment, temper outbursts, and morning headache occur in various combinations. Nighttime symptoms may include sleep talking and walking, enuresis, odd sleeping postures, snorting, and snoring. Marital maladjustment may be a presenting complaint because of loud snoring, restless sleep, loss of libido impotence, and nocturnal enuresis.

The highest frequency of snoring and sleep apnea is reported in the age intervals 0-10 and 40-70 years, the conditions being approximately ten times more common in males.

Different treatments, e.g. uvulo-palatopharyngeo-plastic operations, use of an air blowing pump, or various pharmacological treatments, are used, however with a number of drawbacks. There is still a need for an effective improved treatment of sleep apnea. As regards the sleep, it is known that sleep duration declines gradually and substantially from youth to old age. These age-related sleep changes are a decrease of paradoxical sleep, a decrease in the length of sleep episodes and a decrease in the amplitude of the diurnal rhythm of sleep. There is also a need to improve the quality of sleep in elderly population.

In accordance with the particular findings of the present invention, there is provided:
1.1 A method for the treatment of sleep apnea in a subject in need thereof, comprising administering to said subject a therapeutically effective amount of a somatostatin analogue as hereinbefore defined or a pharmaceutically acceptable salt thereof, e.g. Compound A;
1.2 A method for improving cardiorespiratory function, particularly during sleep, in a subject in need thereof, comprising administering to said subject a therapeutically effective amount of a somatostatin analogue as hereinbefore defined or a pharmaceutically acceptable salt thereof, e.g. Compound A;
1.3 A method for improving airflow in upper airways, particularly during sleep, in a subject in need thereof, comprising administering to said subject a therapeutically effective amount of a somatostatin analogue as hereinbefore defined or a pharmaceutically acceptable salt thereof, e.g. Compound A;
1.4 A method for promoting paradoxical sleep in a subject in need thereof, e.g. in an elderly subject, comprising administering to said subject a therapeutically effective amount of a somatostatin analogue as hereinbefore defined or a pharmaceutically acceptable salt thereof, e.g. Compound A;
2. A somatostatin analogue as hereinbefore defined or a pharmaceutically acceptable salt thereof, e.g. Compound A, for use in any method as defined under 1.1 to 1.4 above;
3. A somatostatin analogue as hereinbefore defined or a pharmaceutically acceptable salt thereof, e.g. Compound A, for use in the preparation of a pharmaceutical composition for use in any method as defined under 1.1 to 1.4 above;
4. A pharmaceutical composition for use in any method as defined under 1.1 to 1.4 above, comprising a somatostatin analogue as hereinbefore defined or a pharmaceutically acceptable salt thereof, e.g. Compound A together with one or more pharmaceutically acceptable diluents or carriers therefor.

Utility of the compounds of the invention, e.g. Compound A in the treatment of disorders, conditions and diseases as hereinbefore specified may be demonstrated for example in accordance with the methods hereinafter described.

A. In vivo Studies

800 to 840-day-old male Wistar rats (Iffa Credo) weighing 670-750 g are implanted under anesthesia with two cortical electrodes and one ground electrode made with chloridized silver wire. After surgery, the rats are housed individually with ad lib water and standard laboratory chow. One week after surgery, the rats are connected to the recording cables and allowed two days for adaptation. Sleep recordings are made from 0906 to 1700 hr on seven days, each separated from the next by an intervening day on which no treatment is given and no recordings are made. All rats receive intraperitoneal injections at 0900 hr in a random fashion, of either saline or a compound of the invention. All measurements of slow wave sleep (SWS) and paradoxical sleep (PS) are made by visual inspection of the polygraph records by two independent observers. For EEG patterns, the following criteria are adopted: Periods of SWS of less than 20 sec within a waking period are not distinguished from waking. Paradoxical sleep is identified only if the event lasts more than 10 sec.

The intraperitoneal administration of a compound of the invention in the rats at a dose of from 0.1 to 0.6 mg/kg results in a selective increase of PS.

B. Clinical Studies

Central Sleep Apnea: 10 patients with central sleep apnea associated with high ventilatory responses to carbon dioxide, are treated with a compound of the invention for 2 months. Sleep recordings, ventilatory control studies (blood gases) and endocrinological controls are performed before, on the first night, at 2 weeks and at 2 months of therapy. In this study, the compounds of the invention, e.g. Compound A, reduce the abnormal high ventilatory responses and the number of central sleep apnea episodes, when administered s.c. at a dose of 100-600 μg.

Obstructive Sleep Apnea: 10 patients with predominantly obstructive sleep apnea are treated with Compound A for 2 months. Sleep recordings, blood gases evaluation and endocrinological controls are performed before, on the first night and at 2 months of therapy. In this study, the compounds of the invention, e.g. Compound A, reduce the number of obstructive sleep apnea episodes when administered s.c. at a dose of 100-600 μg.

For all the above indications the required dosage will of course vary depending upon, for example, the compound used, the host, the mode of administration and the severity of the condition to be treated. In general, however, satisfactory results are obtained by administration in the order of from 0.1 μg to 0.7 mg/kg/day of compound of the invention, e.g. Compound A. An indicated daily dosage for patients is in the range from about 2 μg to about 50 mg, preferably about 0.01 to about 40 mg, e.g. about 0.01 to about 3 mg s.c. of compound of the invention, e.g. Compound A, conveniently administered in divided doses up to 3 times a day in unit dosage form containing for example from about 0.5 μg to about 25 mg, e.g. from about 2 μg to 20 mg, for example from 2 μg to 1.5 mg of the active substance.

The compounds of the invention, e.g. Compound A, may be administered in free form or in pharmaceutically acceptable salt form. Such salts may be prepared in conventional manner and exhibit the same order of activity as the free compound.

The compounds of the invention, e.g. Compound A, may be administered by any conventional route, for example parenterally e.g. in form of injectable solutions or suspensions, orally using a conventional absorption enhancer, in a nasal or a suppository form. The compounds of the invention, e.g. Compound A, may also be administered in sustained release form, e.g. in the form of implants, microcapsules, microspheres or nanospheres comprising e.g. a biodegradable polymer or copolymer, in the form of a liposomal formulation, or in the form of an autogel, e.g. a solid or semi-solid composition capable of forming a gel after interaction with patient's body fluids.

The pharmaceutical compositions may be formulated in conventional manner.

The compounds of the invention, e.g. Compound A, in free from or in pharmaceutically acceptable salt form is well tolerated at dosages required for use in accordance with the present invention.

1-4. (canceled)

5. A method for the treatment of sleep apnea in a subject in need thereof, comprising administering to said subject a therapeutically effective amount of a somatostatin analogue selected from KE108 and a somatostatin analogue of formula I
-(D/L)Trp-Lys-X₁-X₂- I
wherein X₁ is a radical of formula (a) or (b) 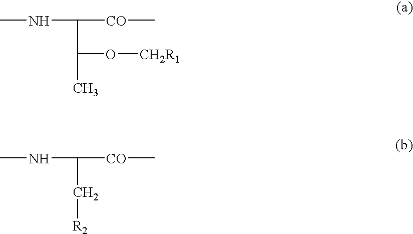
wherein R₁ is optionally substituted phenyl, wherein the substituent may be halogen, methyl, ethyl, methoxy or ethoxy, 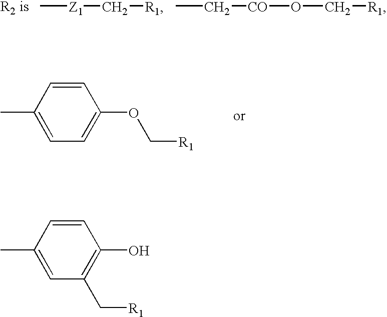
wherein Z₁ is O or S, and
X₂ is an α-amino acid having an aromatic residue on the C_{α} side chain, or an amino acid unit selected from Dab, Dpr, Dpm, His, (Bzl)HyPro, thienyl-Ala, cyclohexyl-Ala and t-butyl-Ala, the residue Lys of said sequence corresponding to the residue Lys⁹ of the native somatostatin-14, or a pharmaceutically acceptable salt thereof.

6. A method for improving cardiorespiratory function, particularly during sleep, in a subject in need thereof, comprising administering to said subject a therapeutically effective amount of a somatostatin analogue as defined in claim 5 or a pharmaceutically acceptable salt thereof.

7. A method for improving airflow in upper airways, particularly during sleep, in a subject in need thereof, comprising administering to said subject a therapeutically effective amount of a somatostatin analogue as defined in claim 5 or a pharmaceutically acceptable salt thereof.

8. A method for promoting paradoxical sleep in a subject in need thereof, e.g. in an elderly subject, comprising administering to said subject a therapeutically effective amount of a somatostatin analogue as defined in claim 5 or a pharmaceutically acceptable salt thereof.

9. A pharmaceutical composition comprising the somatostatin analogue of formula I according to claim 5 or a pharmaceutically acceptable salt thereof, together with one or more pharmaceutically acceptable diluents or carriers therefor.

10. The method according to claim 5, wherein the somatostatin analogue comprising an amino acid sequence of formula I is cyclo[{4-(NH₂—C₂H₄—NH—CO—O—)Pro}-Phg-DTrp-Lys-Tyr(4-Bzl)-Phe] or a pharmaceutically acceptable salt thereof.
